(19) United States
(12) Patent Application Publication
(10) Pub. No.: US 2014/0272970 A1
(43) Pub. Date: Sep. 18, 2014

(54) METHOD FOR QUANTIFYING 5-HYDROXYMETHYLCYTOSINE

(71) Applicant: Promega Corporation, Madison, WI (US)

(72) Inventors: Hicham Zegzouti, Madison, WI (US); Laurie Engel, DeForest, WI (US); Said A. Goueli, Fitchburg, WI (US)

(73) Assignee: Promega Corporation, Madison, WI (US)

(21) Appl. No.: 14/210,785

(22) Filed: Mar. 14, 2014

Related U.S. Application Data
(60) Provisional application No. 61/793,936, filed on Mar. 15, 2013.

(51) Int. Cl. C12Q 1/66

(52) U.S. Cl. CPC C12Q 1/66
USPC 435/6.11

(57) ABSTRACT

Provided herein are methods for detecting and quantifying 5-hydroxymethylated cytosine bases in a DNA molecule.

### CROSS REFERENCE TO RELATED APPLICATIONS

This application claims priority to U.S. Provisional Application No. 61/793,936, filed Mar. 15, 2013, which is incorporated herein by reference in its entirety

### FIELD OF THE INVENTION

The present invention relates to the development of methods for detecting and quantifying 5-hydroxymethylated cytosine bases in a DNA molecule.

### BACKGROUND

Methylation of DNA is catalyzed by DNA methyltransferases and occurs at the 5-carbon position of cytosine residues to form 5-methylcytosines (5-mC). 5-mC constitutes approximately 2-8% of the total cytosines in human genomic DNA and influences a broad range of biological functions. These functions include gene expression, maintenance of genomic integrity, parental imprinting, X-chromosome inactivation, regulation of development, aging and cancer.

Ten-Eleven Translocation proteins (TETs) drive the oxidation of 5-mC. The resultant 5-hydroxymethylcytosines (5-hmC) may play critical roles in the passive/active demethylation of nucleic acid and in the self-renewal and maintenance of embryonic stem cells. Further, the changing levels of 5-hmC in various disease states, for example cancer, indicate that it may influence the disease and could be used as a biomarker for various malignancies.

To study these epigenetic and disease markers, techniques have been developed to either quantify the global levels of 5-hmC in different tissues or disease states or to analyze the 5-mC/5-hmC modifications at specific loci. Unfortunately, these techniques are often cumbersome as they rely on the use of antibodies in multi-step ELISA techniques, on the use of multiple washes and/or the use of radioactivity. Accordingly, there is a need for homogeneous, non-radioactive methods for detecting 5-hmC and/or 5-mC.

### SUMMARY OF THE INVENTION

The present invention is directed to methods for detecting or determining the presence or amount of 5-hmC residues in a DNA. The method may comprise contacting a DNA with a β-Glucosyltransferase (β-GT) and uridine diphospho-glucose (UDP-glucose) to form a first reaction mixture, wherein 5-hmC residues are glucosylated. The first reaction mixture may then be contacted with ADP, a uridine/cytidine monophosphate kinase (CMK), and a buffer to form a second reaction mixture. The buffer may contain a bioluminescent enzyme and a luciferin substrate. The bioluminescent enzyme may be luciferase. The luciferase may be a recombinant luciferase. The luciferase or recombinant luciferase may be thermostable and/or chemostable. The ADP, CMK and buffer may be mixed prior to contact with the first reaction mixture. The mixture of ADP, CMK and buffer may form a UDP detection reagent. Luminescence may be detected in the second reaction mixture, thereby detecting or determining the presence or amount of 5-hmC residues in the DNA. The DNA may be substantially pure. The luminescence generated from the reaction mixture is proportional to the level of glucosylated 5-hmC. The level of glucosylated 5-hmC corresponds to the level of 5-hmC in the DNA. The first reaction mixture may be incubated for an amount of time that is sufficient to allow all of the 5-hmC residues to be glucosylated.

The present invention is also directed to a kit comprising a β-GT, a UDP-glucose, ADP, CMK and a buffer. The buffer may comprise a bioluminescent enzyme and a luciferin substrate. One or more of the β-GT, a UDP-glucose, ADP, CMK and buffer components of the kit may be housed in one or more containers. The kit may include instructions for using the kit to detect or determine the presence or amount of 5-hmC residues in a DNA.

The present invention is further directed to a method for detecting or determining the presence or amount of 5-methylcytosine (5-mC) residues in a DNA. The method may comprise splitting the DNA into a first sample and a second sample. The first sample may be contacted with a 5-mC hydroxylase to form a first reaction mixture, wherein all 5-mC residues are hydroxylated to form 5-hmC. The first reaction mixture may then be contacted with β-GT and UDP-glucose to form a second reaction mixture, wherein all 5-hmC residues are glucosylated. The second reaction mixture may be contacted with ADP, CMK, and a buffer to form a second reaction mixture, wherein the buffer comprises a bioluminescent enzyme and a luciferin substrate. Luminescence may be detected in the reaction mixture, thereby detecting or determining the presence or amount of 5-hmC residues in the first DNA sample. The second sample may be contacted with β-GT and UDP-glucose to form a first reaction mixture, wherein 5-hmC residues are glucosylated. The first reaction mixture may then be contacted with ADP, a CMK, and a buffer to form a second reaction mixture. The buffer may contain a bioluminescent enzyme and a luciferin substrate. Luminescence in the reaction mixture may then be detected, thereby detecting or determining the presence or amount of 5-hmC residues in the DNA in the second sample. The number of relative light units (RLUs) in the second sample may then be subtracted from the number of RLUs in the first sample, wherein the difference in luminescence between the second sample and the first sample corresponds to the presence or an amount of 5-mC residues in the DNA. The luminescence generated from the second reaction mixture in the first sample is proportional to the level of glucosylated 5-hmC in the first sample. The luminescence generated from the second reaction mixture in the second sample is proportional to the level of glucosylated 5-hmC in the second sample. The method steps as they relate to the first sample may be conducted simultaneously with the method steps relating to the second sample. The 5-mC hydroxylase may be one or more of TET1, TET2, and/or TET3. The bioluminescent enzyme may be luciferase. The luciferase may be a recombinant luciferase. The luciferase or recombinant luciferase may be thermostable and/or chemostable. The ADP, CMK and buffer may be mixed prior to contact with the first reaction mixture. The mixture of ADP, CMK and buffer may form a UDP detection reagent. The DNA may be substantially pure.

The present invention is also directed to a method of diagnosing a subject as having a disease, such as cancer, wherein the disease is characterized by an increase or decrease in 5-hmC as compared to a normal control. The method may comprise obtaining a DNA sample from a subject and contacting the DNA with a (β-Glucosyltransferase (β-GT) and uridine diphospho-glucose (UDP-glucose) to form a first reaction mixture, wherein 5-hmC residues are glucosylated. The first reaction mixture may then be contacted with ADP, a uridine/cytidine monophosphate kinase (CMK), and a buffer to form a second reaction mixture. The buffer may contain a bioluminescent enzyme and a luciferin substrate. The bioluminescent enzyme may be luciferase. The luciferase may be a recombinant luciferase. The luciferase or recombinant luciferase may be thermostable and/or chemostable. The ADP, CMK and buffer may be mixed prior to contact with the first reaction mixture. The mixture of ADP, CMK and buffer may form a UDP detection reagent. Luminescence may then be detected in the second reaction mixture, thereby detecting or determining the presence or amount of 5-hmC residues in the DNA. The DNA may be substantially pure. The luminescence generated from the reaction mixture is proportional to the level of glucosylated 5-hmC. The level of glucosylated 5-hmC corresponds to the level of 5-hmC in the DNA. The first reaction mixture may be incubated for an amount of time that is sufficient to allow all of the 5-hmC residues to be glucosylated. The level of the 5-hmC in the DNA sample may be compared to a reference level of 5-hmC in a DNA sample from a healthy subject. The subject may be identified as having the disease, such as cancer, if the level of the 5-hmC in the DNA sample is greater or lower than the reference level of 5-hmC. The method may further comprise administering a treatment regimen to the subject identified as having the disease. The DNA sample from the subject may be from a cancer cell.

The present invention is also directed to a method of diagnosing a subject as having a disease associated with hypo-methylation or hyper-methylation of genomic cytosines. The method may comprise obtaining a DNA sample from a subject and splitting the DNA into a first sample and a second sample. The first sample may be contacted with a 5-mC hydroxylase to form a first reaction mixture, wherein all 5-mC residues are hydroxylated to form 5-hmC. The first reaction mixture may then be contacted with β-GT and UDP-glucose to form a second reaction mixture, wherein all 5-hmC residues are glucosylated. The second reaction mixture may be contacted with ADP, CMK, and a buffer to form a second reaction mixture, wherein the buffer comprises a bioluminescent enzyme and a luciferin substrate. Luminescence may be detected in the reaction mixture, thereby detecting or determining the presence or amount of 5-hmC residues in the first DNA sample. The second sample may be contacted with β-GT and UDP-glucose to form a first reaction mixture, wherein 5-hmC residues are glucosylated. The first reaction mixture may then be contacted with ADP, a CMK, and a buffer to form a second reaction mixture. The buffer may contain a bioluminescent enzyme and a luciferin substrate. Luminescence in the reaction mixture may then be detected, thereby detecting or determining the presence or amount of 5-hmC residues in the DNA in the second sample. The number of relative light units (RLUs) in the second sample may then be subtracted from the number of RLUs in the first sample, wherein the difference in luminescence between the second sample and the first sample corresponds to the presence or an amount of 5-mC residues in the DNA. The luminescence generated from the second reaction mixture in the first sample is proportional to the level of glucosylated 5-hmC in the first sample. The luminescence generated from the second reaction mixture in the second sample is proportional to the level of glucosylated 5-hmC in the second sample. The amount of 5-mC residues in the DNA may be compared to the DNA in a reference control DNA sample from a normal or healthy subject. If the amount or level of the 5-mC in the DNA sample is different that the control DNA, then the subject has a disease associated with hypo-methylation or hyper-methylation of genomic cytosines, such as cancer. The disease may be heart disease, stroke, and/or cancer. The DNA sample from the subject may be from a cancer cell. The method may further comprise administering a treatment regimen to the subject identified as having the disease. The method steps as they relate to the first sample may be conducted simultaneously with the method steps relating to the second sample. The 5-mC hydroxylase may be one or more of TET1, TET2, and/or TET3. The bioluminescent enzyme may be luciferase. The luciferase may be a recombinant luciferase. The luciferase or recombinant luciferase may be thermostable and/or chemostable. The ADP, CMK and buffer may be mixed prior to contact with the first reaction mixture. The mixture of ADP, CMK and buffer may form a UDP detection reagent. The DNA may be substantially pure.

The present invention is further directed to determining whether a compound modulates the hydroxylation of 5-mC in a cell. The method may comprise contacting a cell with a compound. The method may also comprise contacting another cell with a vehicle, such as DMSO, as a control. DNA from the cells may be contacted with a β-Glucosyltransferase (β-GT) and uridine diphospho-glucose (UDP-glucose) to form a first reaction mixture, wherein 5-hmC residues are glucosylated. The first reaction mixture may then be contacted with ADP, a uridine/cytidine monophosphate kinase (CMK), and a buffer to form a second reaction mixture. The buffer may contain a bioluminescent enzyme and a luciferin substrate. The bioluminescent enzyme may be luciferase. The luciferase may be a recombinant luciferase. The luciferase or recombinant luciferase may be thermostable and/or chemostable. The ADP, CMK and buffer may be mixed prior to contact with the first reaction mixture. The mixture of ADP, CMK and buffer may form a UDP detection reagent. Luminescence may then be detected in the second reaction mixture, thereby detecting or determining the presence or amount of 5-hmC residues in the DNA. The DNA may be substantially pure. The luminescence generated from the reaction mixture is proportional to the level of glucosylated 5-hmC. The level of glucosylated 5-hmC corresponds to the level of 5-hmC in the DNA. The first reaction mixture may be incubated for an amount of time that is sufficient to allow all of the 5-hmC residues to be glucosylated. The amount of 5-hmC residues in DNA from the cell contacted with compounds and DNA from the control may be compared. If the amount of the 5-hmC in the DNA sample from the compound-treated cell is different from the 5-hmC in the control DNA, then the compound modulates the hydroxylation of 5-mC.

The present invention is also directed to a method for determining whether a compound modulates the hydroxylation of 5-mC in a DNA. The method may comprise splitting the DNA into a first sample and a second sample. The first sample may be contacted with a 5-mC hydroxylase and a vehicle to form a first reaction mixture. The vehicle may be dimethyl sulfoxide (DMSO). The first reaction mixture may then be contacted with a β-GT and UDP-glucose to form a second reaction mixture, wherein all 5-hmC residues are glucosylated. The second reaction mixture may then be contacted with ADP, CMK, and a buffer to form a second reaction mixture, wherein the buffer may comprise a bioluminescent enzyme and a luciferin substrate. Luminescence may be detected in the reaction mixture, thereby detecting or determining the presence or amount of 5-hmC residues in the DNA in the first sample. The second sample may be contacted with a 5-mC hydroxylase and a compound to form a first reaction mixture. The first reaction mixture may be contacted with a β-GT and a UDP-glucose to form a second reaction mixture, wherein all 5-hmC residues are glucosylated. The second reaction mixture may be contacted with ADP, a CMK, and a buffer to form a second reaction mixture, wherein the buffer may comprise a bioluminescent enzyme and a luciferin substrate. The luminescence in the reaction mixture may be detected, thereby detecting or determining the presence or amount of 5-hmC residues in the DNA in the second sample. If there is a difference in luminescence between the first sample and the second sample, then the compound modulates the hydroxylation of 5-mC in DNA.

The present invention is also directed to method for detecting or determining the presence or amount of 5-hmC residues in a DNA. The method may comprise contacting the DNA with a β-GT and UDP-glucose to form a first reaction mixture, wherein 5-hmC residues are glucosylated. The level of UDP present in the reaction mixture may be detected and determined, thereby detecting or determining the presence or amount of 5-hmC residues in the DNA.

The present invention is also directed to a method for detecting or determining the presence or amount of 5-mC residues in a DNA. The method may comprise splitting the DNA into a first sample and a second sample. The first sample may be contacted with a 5-mC hydroxylase to form a first reaction mixture, wherein all 5-mC residues are hydroxylated to form 5-hmC. The first reaction mixture may be contacted with a β-GT and UDP-glucose to form a second reaction mixture, wherein all 5-hmC residues are glucosylated. The level of UDP present in the reaction mixture may be detected and determined thereby detecting or determining the presence or amount of 5-hmC residues in the DNA in the first sample. The second sample may be contacted with a β-GT and UDP-glucose to form a first reaction mixture, wherein 5-hmC residues are glucosylated. The level of UDP present in the reaction mixture may be detected and determined thereby detecting or determining the presence or amount of 5-hmC residues in the DNA in the second sample. The level of UDP in the second sample may be subtracted from the level of UDP in the first sample, wherein the difference in the level of UDP between the second sample and the first sample corresponds to the presence or an amount of 5-mC residues in the DNA.

### BRIEF DESCRIPTION OF THE DRAWINGS

FIG. 1 illustrates the 5-hydroxymethylcytosine (5-hmC) detection method of the present invention wherein the 5-hmC on a DNA to be analyzed is first glucosylated by β-Glucosyltransferase (β-GT) using UDP-Glucose as a substrate (UDP-Glu). The second step of the method consists of detecting UDP produced from the reaction using a luminescent UDP detection assay.

FIG. 2 illustrates the 5-methylcytosine (5-mC) detection method of the present invention wherein a DNA to be analyzed is split into two equal amounts. One aliquot is subjected to 5-hmC detection method using β-Glucosyltransferase and UDP detection which generates luminescence (RLUs), 5-hmC RLUs. The second aliquot of DNA is first subjected to a TET or oxygenase enzyme reaction to convert all 5-mC on the DNA to 5-hmC; then, all the 5-hmCs are detected using β-Glucosyltransferase and UDP detection method. This will generate luminescence (RLUs), Global RLUs. The amount of light generated from 5-mC is the difference between Global RLUs and 5-hmC RLUs.

FIG. 3 illustrates the linearity of the 5-hmC detection method of the present invention, and the specificity towards 5-hmC DNA. β-Glucosyltransferase was only able to glucosylate the hydroxymethylated DNA, but not the unmethylated or the methylated DNA.

FIG. 4 illustrates the sensitivity of the 5-hmC detection method of the present invention. High signal to background levels were generated with amounts of 5-hmC from low to high pmols.

FIG. 5 illustrates the correlation between the 5-hmC content of DNA and the UDP produced by β-Glucosyltransferase. The amount of 5-hmC was calculated by converting luminescence (RLUs) generated by the UDP detection method to μM amounts using a UDP standard curve.

FIG. 6 illustrates the detection of 5-mC using the conversion of 5-mC to 5-hmC by a TET enzyme and then detecting the 5-hmC using the bioluminescent 5-hmC detection method of the present invention.

FIG. 7 illustrates the quantification of 5-hmC amounts in genomic DNA extracted from two tissue types, brain and spleen.

### DETAILED DESCRIPTION

Reactions governed by certain glycosyltransferases result in the transfer of sugar to an acceptor substrate and the release of uridine diphosphate (UDP) as a universal reaction product. For example, UDP is produced when β-glycosyltransferase (β-GT) catalyzes the glucosylation of 5-hmC residues via uridine diphosphoglucose (UDP-Glu) as a substrate.

On the basis of this biology, the inventors have discovered that detecting or determining the presence or amount of 5-hmC residues in a DNA may be accomplished without the use of antibodies or radioactivity. Central to the herein described methods is the conversion of uridine diphosphate (UDP) to measurable luminescence, which is detected as relative light units (RLUs) and is proportional to the UDP concentration that is produced in β-glycosyltransferase-catalyzed reactions. 5-hmC detection may be accomplished in as little as a single step, in which there is no sample processing, no washing and no use of antibodies or radioactivity. These homogeneous methods are fast, sensitive and simple. The methods are convenient and can be used with any instrumentation platform. Reagents required can be designed with relative ease and may be synthesized readily. The reagents may facilitate measurement of activity in many samples in a high throughput format over a long period of time due to the high signal stability generated by a luminogenic reaction, thus eliminating the need for luminometers with reagent injectors and allowing for batch-mode processing of multiple samples. The present methods can be performed in a single well in a multi-well plate making them suitable for use as high throughput screening methods.

The herein described methods may also provide useful prognostic information related to the hydroxymethylation status of genomic DNA in a subject. The role of hypermethylation in cancer is described in WO 2010/037001. Detection data may be quantified and compared with data that is retrieved from a database over a network or at a computer station. The quantified data may be evaluated in view of retrieved data and a medical condition determined. Accordingly, the present invention is a critical step in the formation of new platforms directed toward the newly transformed glycobiology field where there is a need for new technologies to study the science behind various glycotransferase systems, cancer and disease, and epigenetics.

### 1. DEFINITIONS

The terminology used herein is for the purpose of describing particular embodiments only and is not intended to be limiting. As used in the specification and the appended claims, the singular forms “a,” “and” and “the” include plural references unless the context clearly dictates otherwise.

The term “luminescent,” as used herein, includes bio-luminescence (i.e., light produced by a living organism). When the enzyme involved has evolved in an organism by natural selection for the purpose of generating light, or the enzyme involved is a mutated derivative of such an enzyme, the luminescent reactions are also called “bioluminescent reactions” and the enzyme involved is also called a “bioluminescent enzyme.” Examples of bioluminescent enzymes include, without limitation, beetle luciferase, e.g., firefly luciferase, and the like.

The term “luminogenic molecule” as used herein refers to a molecule capable of creating light via a chemical or biochemical reaction (e.g., luciferin or a functional analog thereof). Suitable luminogenic molecules or substrates for luciferase enzymes include luciferin and functional analogs of luciferins. In some embodiments, functional analogs of luciferins include modified luciferins including derivatives of these compounds. Exemplary compounds include those disclosed in US published application 2009-0075309.

Generally, a luminogenic molecule is either a high energy molecular species (e.g., a stabilized dioxetane), or it is transformed into a high energy molecular species by a chemical reaction. The chemical reaction is usually oxidation by oxygen, superoxide, or peroxide. In each case, the energy within the luminogenic molecule is released by the chemical reaction.

The term “luciferin derivative” as used herein refers to a type of luminogenic molecule or compound having a substantial structure of D-luciferin and is a luciferase substrate, e.g., aminoluciferin, or luciferase substrates disclosed in U.S. application Ser. No. 11/444,145, Branchini et al. (1989), e.g., naphthyl and quinolyl derivatives, Branchini et al. (2002), and Branchini (2000), the disclosures of which are incorporated by reference herein.

“Modulate” as used herein may mean any altering of activity, such as regulate, down regulate, upregulate, reduce, inhibit, increase, decrease, deactivate, or activate.

“Nucleic acid fragment” as used herein may mean a nucleic acid which may be employed at any length, with the total length being limited by the ease of preparation and use in the intended recombinant DNA protocol. Illustrative nucleic acid segments may be useful with total lengths of about 10,000, about 5000, about 3000, about 2,000, about 1,000, about 500, about 200, about 100, about 50 base pairs in length, and the like.

“Small molecules” as used herein may mean a molecule usually less than about 10 kDa molecular weight. Small molecules may be synthetic organic or inorganic compounds, peptides, (poly)nucleotides, (oligo)saccharides and the like. Small molecules specifically include small non-polymeric (i.e. not peptide or polypeptide) organic and inorganic molecules. Many pharmaceutical companies have extensive libraries of such molecules, which may be conveniently screened by using the herein described methods. Small molecules may have molecular weights of less than about 1000 Da, about 750 Da, or about 500 Da.

The term “substantially pure polypeptide” means a polypeptide preparation which contains at the most 10% by weight of other polypeptide material with which it is natively associated (lower percentages of other polypeptide material are preferred, e.g. at the most 8% by weight, at the most 6% by weight, at the most 5% by weight, at the most 4% at the most 3% by weight, at the most 2% by weight, at the most 1% by weight, and at the most ½% by weight). Thus, it is preferred that the substantially pure polypeptide is at least 92% pure, i.e. that the polypeptide constitutes at least 92% by weight of the total polypeptide material present in the preparation, and higher percentages are preferred such as at least 94% pure, at least 95% pure, at least 96% pure, at least 96% pure, at least 97% pure, at least 98% pure, at least 99%, and at the most 99.5% pure. The polypeptides disclosed herein are preferably in a substantially pure form. In particular, it is preferred that the polypeptides disclosed herein are in “essentially pure form,” i.e. that the polypeptide preparation is essentially free of other polypeptide material with which it is natively associated. This can be accomplished, for example, by preparing the polypeptide by means of well-known recombinant methods. Herein, the term “substantially pure polypeptide” is synonymous with the terms “isolated polypeptide” and “polypeptide in isolated form.”

A “substantially pure nucleic acid,” e.g., a substantially pure DNA, etc., is a nucleic acid which is one or both of 1) not immediately contiguous with either one or both of the sequences, e.g., coding sequences, with which it is immediately contiguous (i.e., one at the 5′ end and one at the 3′ end) in the naturally-occurring genome of the organism from which the nucleic acid is derived; or 2) which is substantially free of a nucleic acid sequence with which it occurs in the organism from which the nucleic acid is derived. The term includes, for example, a recombinant DNA which is incorporated into a vector, e.g., into an autonomously replicating plasmid or virus, or into the genomic DNA of a prokaryote or eukaryote, or which exists as a separate molecule (e.g., a cDNA or a genomic DNA fragment produced by PCR or restriction endonuclease treatment) independent of other DNA sequences.

For the recitation of numeric ranges herein, each intervening number there between with the same degree of precision is explicitly contemplated. For example, for the range of 6-9, the numbers 7 and 8 are explicitly contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

### 2. METHOD FOR QUANTIFYING 5-HYDROXYMETHYL-CYTOSINE (5-HMC) AND 5-METHYL-CYTOSINE (5-MC) RESIDUES IN A DNA

The herein described method generates measurable luminescence, wherein the luminescence (relative light units; RLUs) measured is proportional to the level of 5-hmC in a DNA. The method includes contacting a DNA with β-GT and UDP-glucose, whereby 5-hmC residues in the DNA may be glucosylated. The UDP that is produced via the glucosylation of 5-hmC is converted to light-generating ATP by the addition of ADP, a uridine monophosphate/cytidine monophosphate kinase (CMK), and a buffer, which contains a bioluminescent enzyme and a corresponding luciferin substrate. Because a key to the herein described methods is the link between UDP detection and 5-hmC quantification, any method to detect UDP may be used to quantify 5-hmC. For example, as described herein, the conversion of uridine diphosphate (UDP) to measurable luminescence, which is detected as relative light units (RLUs), is one way to ascertain the UDP concentration that is produced in β-glycosyltransferase-catalyzed reactions and the corresponding level of 5-hmC in the DNA.

The glucosylation of 5-hmC residues, the generation of ATP, and the production of ATP-derived luminescence may be performed in a single step. Alternatively, the glucosylation of 5-hmC residues, the generation of ATP, and the production of ATP-derived luminescence may be performed in three separate steps. Alternatively, the glucosylation of 5-hmC residues and the generation of ATP may be performed in a single step, and the production of ATP-derived luminescence may be performed in a separate step. Alternatively, the glucosylation of 5-hmC residues may be performed in a separate step, and the generation of ATP and the production of ATP-derived luminescence may be performed in a single step.

Any one of the reactions involving any of the enzymes may be restricted or limited by time, enzyme concentration, substrate concentration, and/or template concentration. Reaction conditions may be adjusted so that the reaction is carried out under conditions that result in about, at least about, or at most about 20, 30, 40, 50, 60, 70, 80, 90, 95, 96, 97, 98, 99 or 100% completion, or any range derivable therein. For example, the β-GT catalysis of 5-hmC glucosylation on a DNA may be carried out at between 23° C. and 40° C., between 25° C. and 35° C., between 30° C. and 40° C., between 35° C. and 45° C., or between 35° C. and 45° C. The β-GT catalysis of 5-hmC glucosylation on a DNA may be carried out at 34° C., 35° C., 36° C., 37° C., 38° C., 39° C., or 40° C. These temperature conditions may be maintained for 5 minutes, 10 minutes, 20 minutes, 30 minutes, 40 minutes, 50 minutes, 60 minutes, 70 minutes, 80 minutes, 90 minutes, or more.

a. β-GT and α-GT

The β-GT catalyzes the chemical reaction in which a β-D-glucosyl residue is transferred from UDP-glucose to a 5-hmC residue in a DNA. The β-GT may be any β-GT. For example, the β-GT may be from a T4 bacteriophage. The β-GT may be fused to a His-tag. Alternatively, the β-GT may be used without a His-tag. The β-GT may be purified using methods well known in the art. The β-GT can be produced recombinantly or may be directly purified (e.g., from a bacterial cell infected with a T-even bacteriophage). His-tagged β-GT may be recombinantly produced.

Enzymes encoded by bacteriophages of the “T even” family also have hydroxymethylcytosine-glucosylating properties and may be used. Enzymes that add glucose in the alpha configuration are called α-glucosyltransferases (α-GT), while those enzymes that add glucose in the beta configuration are called β-glucosyltransferases (β-GT). T2, T4, and T6 bacteriophages encode α-GTs, but only T4 bacteriophages encode β-GT. As with the β-GT, the α-GT may be purified using methods well known in the art. The α-GT can be produced recombinantly or may be directly purified (e.g., from a bacterial cell infected with a T-even bacteriophage). The α-GT may be fused to a His-tag. Alternatively, the α-GT may be used without a His-tag. The His-tagged α-GT may be recombinantly produced.

In some embodiments, enzymes encoded by bacteriophages of the “T even” family add two glucose molecules linked in a beta-1-6 configuration to hydroxymethylcytosine to form gentibiose-containing-hydroxymethylcytosine. In one embodiment, the enzyme is a β-glucosyl-α-glucosyl-transferase. The β-glucosyl-α-glucosyl-transferase may be encoded by a bacteriophage selected from the group consisting of T2 and T6 bacteriophages.

Any UDP-sugar may be used as a substrate in the context of the herein described methods. For example, the method may be performed using a glucosyltransferase that catalyzes the transfer of a sugar residue from a UDP-N-acetylglucosamine (UDP-GlcNAc) or UDP-galactose (UDP-Gal) to 5-hmC residues in the DNA.

b. Ten-Eleven Translocation Proteins (TETs)

The 5-hydroxymethylcytosine may be naturally occurring. In some embodiments, the 5-hydroxymethylcytosine occurs through contacting DNA with a catalytically active TET family enzyme, a functional TET family derivative or a TET catalytically active fragment thereof, thereby converting methylcytosine to hydroxymethylcytosine. The TET family enzyme may be TET1, TET2, TET3 or CXXC4 protein.

As defined herein, a “naturally occurring” 5-hydroxymethylcytosine residue is one which is found in a sample in the absence of any external manipulation, or activity. For example, a “naturally occurring 5-hydroxymethylcytosine residue” is one found in an isolated DNA that is present due to normal genomic activities, such as, for example, gene silencing mechanisms.

c. DNA

DNA samples that can be used in the method include, without limitation, mammal DNA such as a rodent, mouse, rat, rabbit, guinea pig, ungulate, horse, sheep, pig, goat, cow, cat, dog, primate, human or non-human primate. Plant DNA may also be analyzed according to the invention. For example, DNA from Arabidopsis thaliana, potato, tomato, maize, sorghum, oat, wheat, rice, canola, or soybean may be analyzed. It is further contemplated that genomic DNA from any other organisms containing 5-hmC modification in DNA may be analyzed.

As indicated above, DNA such as genomic DNA can be isolated from one or more cells, bodily fluids or tissues. An array of methods can be used to isolate DNA from samples such as blood, sweat, tears, lymph, urine, saliva, semen, cerebrospinal fluid, feces or amniotic fluid. DNA can also be obtained from one or more cell or tissue in primary culture, in a propagated cell line, a fixed archival sample, forensic sample or archeological sample. Methods for isolating genomic DNA from a cell, fluid or tissue are well known in the art.

Exemplary cell types from which DNA can be obtained in a method of the invention include, a blood cell such as a B lymphocyte, T lymphocyte, leukocyte, erythrocyte, macrophage, or neutrophil; a muscle cell such as a skeletal cell, smooth muscle cell or cardiac muscle cell; germ cell such as a sperm or egg; epithelial cell; connective tissue cell such as an adipocyte, fibroblast or osteoblast; neuron; astrocyte; stromal cell; kidney cell; pancreatic cell; liver cell; or keratinocyte. A cell from which genomic DNA is obtained can be at a particular developmental level including, for example, a hematopoietic stem cell or a cell that arises from a hematopoietic stem cell such as a red blood cell, B lymphocyte, T lymphocyte, natural killer cell, neutrophil, basophil, eosinophil, monocyte, macrophage, or platelet. Other cells include a bone marrow stromal cell (mesenchymal stem cell) or a cell that develops therefrom such as a bone cell (osteocyte), cartilage cells (chondrocyte), fat cell (adipocyte), or other kinds of connective tissue cells such as one found in tendons; neural stem cell or a cell it gives rise to including, for example, a nerve cells (neuron), astrocyte or oligodendrocyte; epithelial stem cell or a cell that arises from an epithelial stem cell such as an absorptive cell, goblet cell, Paneth cell, or enteroendocrine cell; skin stem cell; epidermal stem cell; or follicular stem cell. Generally any type of stem cell can be used including, without limitation, an embryonic stem cell, adult stem cell, totipotent stem cell or pluripotent stem cell.

A cell from which a genomic DNA sample is obtained for use in the invention can be a normal cell or a cell displaying one or more symptom of a particular disease or condition. Thus, a genomic DNA used in a method of the invention can be obtained from a cancer cell, neoplastic cell, apoptotic cell, senescent cell, necrotic cell, an autoimmune cell, or a cell comprising a heritable genetic disease, for example. Further, the DNA sample may be obtained from a cell that has been genetically modified via recombinant techniques, whereby the genome of which is augmented with a recombinant DNA. The DNA may encode one or more proteins related to a particular disease. For example, the recombinant DNA may be over-expressed or under-expressed so as to impart a disease phenotype to the cell. The DNA sample may be obtained from a cell that was treated with one or more compounds that are thought to directly or indirectly modulate DNA methylation and/or demethylation processes. Accordingly, the methods described herein may be used to understand DNA methylation and demethylation in the context of disease and/or to study compounds thought to inhibit or activate cellular processes that may be involved in DNA methylation and demethylation.

DNA for use according to the invention may be a standard or reference DNA sample. Such reference samples may comprise a known level of DNA hydroxymethylation. For example, reference DNA samples may be DNA extracted from cells that lack one of more DNA methyltransferase enzyme and are essentially devoid of methylation and hydroxymethylation. A reference DNA sample may be treated with a DNA methyltransferase and an enzyme to convert methylated cytosines into hydroxymethylcytosines (e.g., TET1, TET2 or TET3, see Tahiliani et al., Science, 324:930-935 (2009), incorporated herein by reference) and therefore comprise hydroxymethylation at most or essentially all potential methylation sites. In certain aspects, methods according to the invention involve the use of two or more standard DNA samples, such as DNA samples comprising essentially no methylation and essentially complete methylation.

d. Uridine/Cytidine Monophosphate Kinase (CMK), ADP, and Buffer

CMK is in a class of transferase enzymes that catalyze the reversible formation of ATP and a nucleoside monophosphate from a nucleoside diphosphate and ADP. Accordingly, in one direction, this reaction involves the transfer of a high energy phosphate from ATP to a nucleoside monophosphate to form the corresponding nucleoside diphosphate and ADP; while in the other direction, the reaction involves a transfer of a phosphate from the nucleoside diphosphate to ADP, thereby generating ATP and the corresponding nucleoside monophosphate. CMK is capable of transferring phosphates from UDP to ADP to form ATP.

The ATP that is generated may be detected by any suitable means or method of ATP detection that is specific for ATP, and that hydrolyzes or otherwise destroys or removes the ATP being detected. One such ATP detection method involves the ATP-dependent generation of light by a second reaction catalyzed by luciferase. The ATP will be utilized by the luciferase, along with luciferin and sufficient molecular oxygen (O₂) to drive the detection of ATP, thereby generating AMP, PPi, oxyluciferin, CO₂, and light.

The DNA sample may be depleted of any intrinsic ATP that could interfere with the read out of the detection of ATP. Alternatively, the level of intrinsic ATP may be determined using an ATP detection method of choice so that the amount of intrinsic ATP may be subtracted as background from the targeted reactions described herein.

The reaction mixtures described herein may contain Mg²⁺ in a concentration appropriate for use as a required cofactor of the CMK. In addition, and optionally, the reaction mixtures may contain one or more sufficiently pure nucleotides (i.e., nucleoside triphosphate, nucleoside diphosphate, and/or nucleoside monophosphate) to serve as a substrate for the reaction catalyzed by the CMK. For example, the nucleoside diphosphate may be ADP. Further, the mixtures may contain one or more buffer components common to in vitro enzymatic reactions. Such components may include Tris pH 7.5, NaCl, MgCl₂, and/or dithiothreitol (DTT).

Methods and compositions may involve a purified, or substantially pure, DNA, UDP-Glu, and/or enzyme, such as luciferase, β-glucosyltransferase and TET1, TET2, TET3, or CXXC4. Such protocols are known to those of skill in the art. In certain embodiments, purification may result in a molecule that is about or at least about 70, 75, 80, 85, 90, 95, 96, 97, 98, 99, 99.1, 99.2, 99.3, 99.4, 99.5, 99.6, 99.7 99.8, 99.9% or more pure, or any range derivable therein, relative to any contaminating components (w/w or w/v).

e. Bioluminescent Enzyme and a Corresponding Substrate—Luciferase and Luciferin

Luciferase enzymes produce catalytic products that provide a detectable light product, sensitivity, and allow easy measurement of ATP. However, any bioluminescence generating-enzyme that is ATP-dependent may be used in the methods and compositions of the present invention.

At their most basic level, luciferases are defined by their ability to produce luminescence. More specifically, a luciferase is an enzyme that catalyzes the oxidation of a substrate, luciferin, to produce oxiluciferin and photons.

To date, several classes of luciferases have been identified. Of these, beetle luciferases, such as that of the common firefly (family Lampyridae), form a distinct class with unique evolutionary origins. Beetle luciferases are often referred to as firefly luciferases in the literature; however, firefly luciferases are actually a subgroup of the beetle luciferase class. Beetle luciferases may be purified from the lanterns of the beetles themselves or from protein expression systems well known in the art.

Beetle luciferases, particularly firefly luciferase from the North American firefly Photinus pyralis, are well known in the art. The P. pyralis luciferase (LucPpy) consists of approximately 550 amino acids of Mᵣ 61 kDa as calculated by the protein encoded by the nucleotide sequence of the gene. However, other firefly luciferases are known, such as Photuris pennsylvanica finely luciferase (LucPpe2; 545 amino acid residues; GenBank 2190534). Thermostable and/or chemostable mutant luciferases derived from LucPpe2 (e.g., LucPpe2m78 (also known as 78-0B10); LucPpe2m90 (also known as 90-1B5); LucPpe2m133 (also known as 133-1B2); LucPpe2m146 (also known as 146-1H2) may be employed, however, any luciferase that meets the limitations set forth herein may be used in the composition, method and kits of the invention. The method of making mutant luciferases from LucPpe is disclosed in PCT/US99/30925.

Isolated and/or purified luciferases are typically used in the present invention. Luciferases that may be used in the methods, compositions and kits described herein include those found in WO 1999/14336, WO 2001/20002, EP 1 124 944, EP 1 224 294, U.S. Pat. Nos. 6,171,808, 6,132,983, and 6,265,177.

Luciferases can be isolated from biological specimens that produce luciferase or from a cell that expresses an exogenous polynucleotide encoding a desired luciferase. Such techniques are well known to those of skill in the art (see U.S. Pat. No. 6,602,677).

The naturally-occurring substrate for beetle luciferases is firefly luciferin, a polytherocyclic organic acid, D-(−)-2-(6′-hydroxy-2′-benzoth-iazolyl)-Δ²-thiazolin-4-carboxylic acid (D-luciferin). Luciferin may be isolated from nature (e.g., from fireflies) or synthesized. Synthetic luciferin can have the same structure as the naturally occurring luciferin or can be derivatized, so long as it functions analogously. Examples of derivatives of luciferin include D-luciferin methyl ester and other esters of luciferase that are hydrolyzed or acted upon by esterases in a sample to yield luciferin, and naphthyl- and quinolyl-luciferin (Branchini et al., 1989). There are multiple commercial sources for luciferin (e.g., Promega Corp. Madison, Wis.).

The beetle luciferase-catalyzed reaction that yields luminescence (the luciferase-luciferin reaction) involves firefly luciferin, adenosine triphosphate (ATP), magnesium, and molecular oxygen. In the initial reaction, the firefly luciferin and ATP react to form luciferyl adenylate with the elimination of inorganic pyrophosphate. The luciferyl adenylate remains tightly bound to the catalytic site of luciferase. When this form of the enzyme is exposed to molecular oxygen, the enzyme-bound luciferyl adenylate is oxidized to yield oxyluciferin in an electronically excited state. The excited oxidized luciferin emits light on returning to the ground state.

f. Variant Enzymes

A full length luciferase, CMK, β-GT, TET1, TET2, TET3, or CXXC4 variant will have at least about 80% amino acid sequence identity, at least about 81% amino acid sequence identity, such as at least about 82%, 83%, 84%, 85%, 86%, 87%, 88%, 89%, 90%, 91%, 92%, 93%, 94%, 95%, 96%, 97%, 98%, or 99% amino acid sequence identity with a corresponding full-length native luciferase, CMK, β-GT, TET1, TET2, TET3, or CXXC4 e.g., one retaining the ability to generate luminescence, transfer phosphate groups, transfer glucose, and hydroxylate, respectively. Ordinarily, variant fragments are at least about 50 amino acids in length, often at least about 60 amino acids in length, more often at least about 70, 80, 90, 100, 150, 200, 300, 400, 500 or 550 amino acids in length, or more and retain the ability to generate luminescence, transfer phosphate groups, transfer glucose, and hydroxylate. A full length luciferase, CMK, β-GT, TET1, TET2, TET3, or CXXC4 fragment thereof, or variant thereof may be fused to heterologous amino acid sequences and still be functional in the invention.

For example, full length beetle luciferase, CMK, β-GT, TET1, TET2, TET3, or CXXC4 fragments thereof or variants thereof used in the compositions and methods of the present invention may be purified from a native source or prepared by a number of techniques, including (1) chemical synthesis, (2) enzymatic (protease) digestion of luciferase, and (3) recombinant DNA methods. Chemical synthesis methods are well known in the art, as are methods that employ proteases to cleave specific sites. To produce the enzymes, variant enzymes or fragments thereof, DNA encoding the enzymes, variants and fragments can be prepared and then expressed in a host organism, such as E. coli. Methods such as endonuclease digestion or polymerase chain reaction (PCR) allow one of skill in the art to generate an unlimited supply of well-defined fragments. The activity of a variant or fragment may vary from that of the native enzyme.

Any type of amino acid substitution, insertion or deletion, or combination thereof may be used to generate a variant luciferase, CMK, β-GT, TET1, TET2, TET3, or CXXC4. However, a luciferase, CMK, β-GT, TET1, TET2, TET3, or CXXC4 with a conservative amino acid substitution is more likely to retain activity. Conservative substitutions whereby an amino acid of one class is replaced with another amino acid of the same type fall within the scope of the invention if the substitution does not impair enzyme activity.

Non-conservative substitutions that affect (1) the structure of the polypeptide backbone, such as a β-sheet or a-helical conformation, (2) the charge or (3) hydrophobicity, or (4) the bulk of the side chain of the target site might modify luciferase function. Residues are divided into groups based on common side-chain properties.

Variant luciferase, CMK, β-GT, TET1, TET2, TET3, or CXXC4 genes or gene fragments can be made using methods known in the art such as oligonucleotide-mediated (site-directed) mutagenesis, alanine scanning, and PCR mutagenesis. Site-directed mutagenesis) cassette mutagenesis, restriction selection mutagenesis, PCR mutagenesis or other known techniques can be performed on the cloned DNA to produce the variant DNA.

### 3. METHODS FOR DIAGNOSING DISEASE

The herein described methods for detecting or determining the presence or amount of 5-hmC or 5-mC may be incorporated into methods for diagnosing disease in a subject and further treating the subject. Changes in methylation status have been proposed to inactivate tumor suppressors and activate oncogenes, thereby contributing to tumorigenesis. See, for example, Gal-Yam et al., Annu Rev. Med. (2008)59:267. In certain tissues, such as the pancreas and kidney, hypermethylation is an early event and the number of aberrant hypermethylation events increases progressively from a precancerous to a cancerous state. See, for example, Arai E. et al., Regional DNA hypermethylation and DNA methyltransferase (DNMT) 1 protein overexpression in both renal tumors and corresponding nontumorous renal tissues. Int J Cancer. 2006; 119:288-296; and Peng D F et al., DNA methylation of multiple tumor-related genes in association with overexpression of DNA methyltransferase 1 (DNMT1) during multistage carcinogenesis of the pancreas. Carcinogenesis. 2006; 27:1160-1168. The importance of hypermethylation of genes in cancer has become so well recognized that databases such as PubMeth (www.pubmeth.org) are now available allowing one to search for evidence of methylation of a gene of interest in different cancer types. Further, significant differences of 5-hmC content in different tissues have been observed. See, for example, Li et al., Distribution of 5-Hydroxymethylcytosine in Different Human Tissues, Journal of Nucleic Acids, Volume 2011, Article ID 870726, doi:10.4061/2011/870726. The percentage of 5-hmC is found to be high, for example, in brain, liver, kidney and colorectal tissues (0.40-0.65%), while it is relatively low in lung (0.18%) and very low in heart, breast, and placenta (0.05-0.06%). With respect to cancerous colorectal tissues, the abundance of 5-hmC was significantly reduced (0.02-0.06%) compared to that in normal colorectal tissues (0.46-0.57%). Li et al., Journal of Nucleic Acids, Volume 2011, Article ID 870726, doi:10.4061/2011/870726.

The herein described methods for diagnosing disease, such as cancer, make use of the generation of measurable luminescence, wherein the luminescence (relative light units; RLUs) measured is proportional to the level of 5-hmC in a DNA. The presence or amount of 5-hmC or 5-mC in a DNA obtained from a cell or tissue sample from a subject may be compared to the level of 5-hmC or 5-mC in a reference sample or control sample, whereby any difference in 5-hmC or 5mC levels between the cell or tissue sample from a subject and the reference sample or control sample may indicate that the subject has cancer, for example.

a. Subject

The subject may be a mammal, which may be a human. The subject may have, or be at risk of, developing a cancer or other disease.

b. Control

It may be desirable to include a control in any of the herein described methods. The control may be a sample or a DNA. The control may be analyzed concurrently with the sample or DNA from the subject as described above. The results obtained from the subject sample or DNA can be compared to the results obtained from the control sample. Standard curves may be provided, with which assay results for the biological sample may be compared. Such standard curves present levels of marker as a function of assay units, i.e. luminescent signal intensity. Using samples taken from multiple donors, standard curves can be provided for control levels of 5-hmC or 5-mC in normal tissue, as well as for “at-risk” levels of the 5-hmC or 5-mC in tissue taken from donors.

Control cells may be contacted by the candidate modulator compound and compared with cells comprising the gene knockouts of one or more hydroxylases. The control cells can be used to aid in the identification of modulators from a pool or library of candidates. For example, a positive control cell for identifying a candidate modulator that inhibits a 5-mC hydroxylase may be a cell that fails to express any 5-mC hydroxylase. A negative control may comprise contacting the candidate modulator compound with a cell that constitutively expresses one or more 5-mC hydroxylases, for example. Other controls may include the use of known 5-mC hydroxylase inhibitors such as a 2-hydroxyglutarate. Still other controls may include the use of a vehicle, such as dimethyl sulfoxide (DMSO).

c. Disease and Cancer

The subject may or may not be genetically predisposed to develop a disease. The disease may be characterized by an increase or decrease in 5-hmC or 5-mC as compared to a control. The disease may be characterized by hypo-methylation or hyper-methylation of genomic cytosines. The disease may be, for example, heart disease, stroke, or cancer. The cancer may be colorectal, brain, skin, stomach, prostate, lung, and/or ovarian cancer. The ovarian cancer may be epithelial ovarian cancer (EOC), for example.

### 4. METHODS OF SCREENING FOR MODULATORS OF 5-MC HYDROXYLATION

The herein described methods for detecting or determining the presence or amount of 5-hmC or 5-mC may be incorporated into methods of screening candidate modulators of 5-mC hydroxylation. As discussed above, changes in methylation status have been proposed to inactivate tumor suppressors and activate oncogenes, thereby contributing to tumorigenesis.

The herein described methods of screening for modulators of 5-mC hydroxylation again make use of the generation of measurable luminescence, wherein the luminescence (relative light units; RLUs) measured is proportional to the level of 5-hmC in a DNA. The DNA, or a cell or tissue from which the DNA is derived, may be contacted with a compound of interest. The DNA, cell, or tissue, may be contacted with, for example, a compound, protein, nucleic acid, or small molecule, cell extract, or nuclear extract, concomitant with the 5-mC hydroxylase to form a reaction mixture, wherein 5-mC residues may be hydroxylated to form 5-hmC.

Pursuant to the herein described methods for determining the presence or amount of 5-hmC or 5-mC in a DNA, the level of 5-hmC or 5-mC obtained from a cell or tissue sample from a subject may be compared to the level of 5-hmC or 5-mC in a reference sample or control sample. Any difference in 5-hmC or 5mC levels between the cell or tissue sample from a subject and the reference sample or control sample may indicate that the compound modulates directly or indirectly the hydroxylation of 5-mC.

a. Candidate Modulator

A variety of different types of libraries of candidate modulator compounds can be used and screened in the method of the present invention. A candidate modulator may be an antibody, a small molecule, a drug, a peptide, a nucleic acid, an oligosaccharide, or an inorganic compound. An identified modulator compound may be derived from a library of candidate modulator compounds. A library of compounds may be a combinatorial library. The method may comprise stimulating a host cell to express the candidate modulator compound.

Modulators identified by the herein described method, may be compounds showing pharmacological activity or therapeutic activity. Compounds with pharmacological activity are able to enhance or interfere with the activity of a 5-mC hydroxylase or a fragment thereof. The compounds having the desired pharmacological activity may be administered in a physiologically acceptable carrier to a host.

The agents may be administered in a variety of ways, orally, parenterally e.g., subcutaneously, intraperitoneally, intravascularly, etc. Depending upon the manner of introduction, the compounds may be formulated in a variety of ways. The concentration of a therapeutically active compound in the formulation may vary from about 0.1-100 wt %. Modulators of the present invention can be administered at a rate determined by the LD-50 of the modulator, and the side-effects of the modulator at various concentrations, as applied to the mass and overall health of the subject. Administration can be accomplished via single or divided doses.

The identified modulators of the invention may be used alone or in conjunction with other agents that are known to be beneficial in treating or preventing human diseases that are mediated by DNA methylation, demethylation, and/or 5-mC hydroxylation. The modulators of the invention and another agent may be co-administered, either in concomitant therapy or in a fixed combination, or they may be administered at separate times.

### 5. KIT

Kits for analysis of DNA hydroxymethylation are provided herein. The kit may comprise reagents for analysis of total DNA hydroxymethylation levels by labeling 5′-hmC positions with a glucose. Such kits comprise an active glucosyltransferase, such as β-glucosyltransferase. The kit may be provided for determining one or more hydroxymethylated positions in a DNA sample. Kits according to the invention can further comprise an enzyme that converts 5′-mC into 5′-hmC (e.g., recombinant TET1, TET2, TET3, and/or CXXC4 proteins); one or more reference DNA samples; a glucosylation buffer; UDP-glucose; instructions; a bioluminescent enzyme and a corresponding cognate light-emitting substrate. Suitable kit components, compositions and buffers that may be used in the described methods can also be obtained commercially, e.g. UDP-Glo glycosyltransferase assay from Promega Corporation. For example, the kit components, compositions and buffers may also be modified by the addition of suitable components, including enzymes, such as CMK, components, such as ADP, salts, chelators, etc. The different components may comprise subsets of these parts and may be combined in any way that either facilitates the application of the invention or prolongs storage life.

In some embodiments, the kit comprises a separate container comprising lyophilized luciferase. In some embodiments, the container comprising lyophilized luciferase further comprises lyophilized luciferin or a derivative thereof that is a luciferase substrate.

One or more reagents may be supplied in a solid form or liquid buffer that is suitable for inventory storage, and later for addition into the reaction medium when the method of using the reagent is performed. Suitable packaging is provided.

(1) Containers/Vessels

The reagents included in the kits can be supplied in containers of any sort such that the life of the different components are preserved and are not adsorbed or altered by the materials of the container. For example, sealed glass ampules may contain lyophilized luciferase or buffer that has been packaged under a neutral, non-reacting gas, such as nitrogen. Ampules may consist of any suitable material, such as glass, organic polymers, such as polycarbonate, polystyrene, etc., ceramic, metal or any other material typically employed to hold reagents. Other examples of suitable containers include simple bottles that may be fabricated from similar substances as ampules, and envelopes, that may consist of foil-lined interiors, such as aluminum or an alloy. Other containers include test tubes, vials, flasks, bottles, syringes, or the like. Containers may have a sterile access port such as a bottle having a stopper that can be pierced by a hypodermic injection needle. Other containers may have two compartments that are separated by a readily removable membrane that upon removal permits the components to mix. Removable membranes may be glass, plastic, rubber, etc.

(2) Instructional Materials

The kits may also be supplied with instructional materials. Instructions may be printed on paper or other substrate and/or may be supplied as an electronic-readable medium such as a floppy disc, CD-ROM, DVD-ROM, Zip disc, videotape, audio tape, etc. Detailed instructions may not be physically associated with the kit; instead, a user may be directed to an internet web site specified by the manufacturer or distributor of the kit, or supplied as electronic mail.

### 6. LUMINESCENCE DETECTION

The luminescence generated by a luciferase-luciferin reaction is typically detected with a luminometer although other detection means may be used. The presence of light greater than background level indicates the presence of ATP in the sample. The background level of luminescence is typically measured in the same matrix, but in the absence of the sample. Suitable control reactions are readily designed by one of skill in the art. Luciferases may allow for multiple analyses of a sample over time or analysis of many samples over time. Optionally, the luciferases used in the compositions and methods of the invention have enhanced thermostability and/or chemostability properties.

Quantifying the amount of luminescence also quantifies the amount of ATP, and thus the amount of UDP produced by the β-GT in a sample. The amount of UDP produced is directly proportional to the 5-hmC in the DNA sample. Thus, quantitation of ATP allows for quantitation of UDP and 5-hmC. Quantitative ATP values are realized, for example, when the luminescence generated from a test sample, in which UDP is converted to ATP via the methods of the invention which monitor UDP formation by converting it to ATP, is compared to the luminescence generated from a control sample or to a standard curve determined by using known amounts of ATP and the same luciferase and reaction conditions (i.e., temperature, pH, etc.). It is understood that quantification involves subtraction of background values. Qualitative ATP values are realized when the luminescence generated from one sample is compared to the luminescence generated from another sample without a need to know the absolute amount of ATP converted from UDP present in the samples.

The herein described method may involve comparing the luminescence results to a control or a comparative sample. For example, the control or comparative sample may contain a DNA having a known number or quantity of 5-mC and/or 5-hmC. A standard curve of 5-hmC DNA may be performed to correlate the luminescence (RLUs) with the amount of 5-hmC present in each DNA sample. To compare the samples for content of 5-hmC, luminescence may be converted to μM 5-hmC based on the standard curve, then to pmol amount of 5-hmC in each DNA.

The present invention can be utilized as illustrated by the following non-limiting examples.

### Example 1

### Materials Relating to Examples 2-5

All DNA samples used in the below examples were purchased from Active Motif or ZYMO Research. T4 β-Glucosyltransferase was either purchased from ZYMO Research or recombinantly produced at Promega Corporation. The UDP Detection Reagent contains Promega Glo buffer with UDP converting enzyme (CMK) and luciferase/luciferin component. TET1 enzyme was purchased from Active Motif

### Example 2

### Converting 5-hmC to Luminescence

DNA containing a known amount of cytosines was used in this experiment. DNA with all cytosines unmodified, methylated or hydroxymethylated were serially diluted in multi-well plates starting from 100 ng DNA in 25 μl of total reaction volume. Reactions were performed in GT Buffer (10 mM Tris pH 7.5, 10 mM NaCl, 10 mM MgCl₂ and 1 mM DTT) containing 0.125 U β-GT and 50 mM UDP-glucose donor substrate. The glucosyltransferase reaction was performed at 37° C. for 1 hour. To detect the UDP produced, 25 μl of UDP Detection Reagent was added to convert UDP to ATP and then ATP to light. After an hour, the luminescence generated was measured on a luminometer.

As shown in FIG. 3, the 5-hmC detection signal was linear and proportional to the amount of 5-hmC in the sample. Only the 5-hmC containing DNA was able to be glucosylated by β-GT and hence generated UDP. The unmethylated and methylated cytosines could not be glucosylated by the glycosyltransferase suggesting that the method described in FIG. 1 is very specific to 5-hmC.

FIG. 4 illustrated that the method of the present invention is very sensitive and can detect as low as 1 pmol 5-hmC with more than 2-fold luminescent signal over background using only 100 ng DNA.

### Example 3

### Luminescence is Proportional to UDP and 5-hmC

UDP was titrated in 25 μl GT buffer to create a standard curve. 25 μl of UDP detection reagent was added, and after 1 hour at room temperature, luminescence was measured on a luminometer. Luminescence generated from 5-hmC conversion as described in Example 1 was converted to concentration of UDP formed based on the UDP standard curve.

FIG. 5 shows that luminescence is proportional to the UDP produced, and, by using the UDP standard curve, there is a positive correlation between the amount of 5-hmC DNA and the UDP produced by β-GT.

### Example 4

### 5-Methylcytosine Detection

5-mC containing DNA was incubated with 1 ug TET1 oxygenase enzyme in the presence of GT buffer supplemented with 2 mM ascorbate, 1 mM α-ketoglutarate, 100 μM Ferrous Ammonium Sulfate (Fe²⁺), 0.125 U β-GT and 50 μM UDP-Glucose. The reaction was incubated for 2 hours at 37° C., and the UDP produced detected using the UDP Detection Reagent as described previously.

FIG. 6 shows that the method of the present invention and illustrated in FIG. 2 can detect 5-mC by converting it to 5-hmC and using the 5-hmC detection method described herein.

### Example 5

### Detection of 5-hmC in DNA Extracted from Different Tissue Types

500 ng of DNA extracted from brain or spleen tissue was incubated in GT buffer containing 0.125 U β-GT and 50 μM UDP-Glucose for 1 hour at 37° C. The UDP produced was detected using the UDP Detection Reagent as described previously. A standard curve of 5-hmC DNA was performed at the same time to correlate the luminescence (RLUs) with the amount of 5-hmC present in each DNA sample. To compare the samples for content of 5-hmC, luminescence was converted to μM 5-hmC based on the standard curve, then to pmol amount of 5-hmC in each DNA.

FIG. 7 illustrates that the method of the present invention is capable of detecting global levels of 5-hmC in DNA extracted from different tissue types.

While the present invention is described in connection with what is presently considered to be the most practical and preferred embodiments, it should be appreciated that the invention is not limited to the disclosed embodiments, and is intended to cover various modifications and equivalent arrangements included within the spirit and scope of the claims. Modifications and variations in the present invention may be made without departing from the novel aspects of the invention as defined in the claims. The appended claims should be construed broadly and in a manner consistent with the spirit and the scope of the invention herein.

1. A method for detecting or determining the presence or amount of 5-hydroxymethylcytosine (5-hmC) residues in a DNA, the method comprising:
(a) contacting the DNA with a β-Glucosyltransferase (β-GT) and uridine diphospho-glucose (UDP-glucose) to form a first reaction mixture, wherein 5-hmC residues are glucosylated;
(b) contacting the first reaction mixture with ADP, a uridine/cytidine monophosphate kinase (CMK), and a buffer to form a second reaction mixture, wherein the buffer comprises a bioluminescent enzyme and a luciferin substrate; and detecting luminescence in the second reaction mixture, thereby detecting or determining the presence or amount of 5-hydroxymethylcytosine (5-hmC) residues in the DNA.

2. The method of claim 1, wherein the luminescence generated from the second reaction mixture is proportional to the level of glucosylated 5-hmC.

3. The method of claim 2, wherein the level of glucosylated 5-hmC corresponds to the level of 5-hmC in the DNA.

4. The method of claim 1, wherein the first reaction mixture is incubated for an amount of time sufficient to allow all of the 5-hmC residues to be glucosylated.

5. The method of claim 1, wherein the bioluminescent enzyme is luciferase.

6. (canceled)

7. (canceled)

8. The method of claim 1, wherein the ADP, CMK, and buffer mixed prior to contact with the first reaction mixture (thereby forming the UDP detection reagent).

9. (canceled)

10. (canceled)

11. (canceled)

12. A method for detecting or determining the presence or amount of 5-methylcytosine residues (5-mC) in a DNA, the method comprising:
(a) splitting the DNA into a first sample and a second sample;
(b) contacting the first sample with a 5-mC hydroxylase to form a first reaction mixture, wherein all 5-mC residues are hydroxylated to form 5-hmC;
(c) contacting the first reaction mixture with a β-Glucosyltransferase (β-GT) and uridine diphospho-glucose (UDP-glucose) to form a second reaction mixture, wherein all 5-hmC residues are glucosylated;
(d) contacting the second reaction mixture with ADP, a uridine/cytidine monophosphate kinase (CMK), and a buffer to form a second reaction mixture, wherein the buffer comprises a bioluminescent enzyme and a luciferin substrate;
(e) detecting luminescence in the reaction mixture, thereby detecting or determining the presence or amount of 5-hydroxymethylcytosine (5-hmC) residues in the DNA in the first sample;
(f) contacting the second sample with a β-Glucosyltransferase (β-GT) and uridine diphospho-glucose (UDP-glucose) to form a first reaction mixture, wherein 5-hmC residues are glucosylated;
(g) contacting the first reaction mixture with ADP, a uridine/cytidine monophosphate kinase (CMK), and a buffer to form a second reaction mixture, wherein the buffer comprises a bioluminescent enzyme, and a luciferin substrate;
(h) detecting luminescence in the reaction mixture, thereby detecting or determining the presence or amount of 5-hydroxymethylcytosine (5-hmC) residues in the DNA in the second sample; subtracting the number of relative light units in the second sample from the number of relative light units in the first sample, wherein the difference in luminescence between the second sample and the first sample corresponds to the presence or an amount of 5-methylcytosine (5-mC) residues in the DNA.

13. The method of claim 12, wherein the luminescence generated from the second reaction mixture in the first sample is proportional to the level of glucosylated 5-hmC in the first sample.

14. The method of claim 12, wherein the luminescence generated from the second reaction mixture in the second sample is proportional to the level of glucosylated 5-hmC in the second sample.

15. The method of claim 12, wherein steps (c)-(e) are conducted simultaneously with steps (f)-(h).

16. (canceled)

17. The method of claim 12, wherein the bioluminescent enzyme is luciferase.

18. (canceled)

19. (canceled)

20. The method of claim 12, wherein the ADP, CMK, and buffer mixed prior to contact with the first reaction mixture (UDP detection reagent).

21. (canceled)

22. (canceled)

23. (canceled)

24. (canceled)

25. (canceled)

26. (canceled)

27. (canceled)

28. (canceled)

29. A method for determining whether a compound modulates the hydroxylation of 5-mC in a cell, the method comprising:
(a) contacting a cell with the compound;
(b) contacting another similar cell with a vehicle as a control;
(c) obtaining a DNA sample from both treated and control cells;
(d) performing the method of claim 1 on the DNA samples and comparing the amount of 5-hmC residues in both DNA samples, wherein if the amount of the 5-hmC in the DNA sample from the compound treated cell is different from the control DNA, the compound modulates the hydroxylation of 5-mC.

30. A method for determining whether a compound modulates the hydroxylation of 5-mC in a DNA, the method comprising:
(a) splitting the DNA into a first sample and a second sample;
(b) contacting the first sample with a 5-mC hydroxylase and a vehicle to form a first reaction mixture;
(c) contacting the first reaction mixture with a β-GT and UDP-glucose to form a second reaction mixture, wherein all 5-hmC residues are glucosylated;
(d) contacting the second reaction mixture with ADP, a CMK, and a buffer to form a second reaction mixture, wherein the buffer comprises a bioluminescent enzyme and a luciferin substrate;
(e) detecting luminescence in the reaction mixture, thereby detecting or determining the presence or amount of 5-hmC residues in the DNA in the first sample;
(f) contacting the second sample with a 5-mC hydroxylase and a compound to form a first reaction mixture;
(g) contacting the first reaction mixture with a β-GT and a UDP-glucose to form a second reaction mixture, wherein all 5-hmC residues are glucosylated;
(h) contacting the second reaction mixture with ADP, a CMK, and a buffer to form a second reaction mixture, wherein the buffer comprises a bioluminescent enzyme and a luciferin substrate;
(i) detecting luminescence in the reaction mixture, thereby detecting or determining the presence or amount of 5-hmC residues in the DNA in the second sample;
(j) wherein, if there is a difference in luminescence between (i) and (e), then the compound modulates the hydroxylation of 5-mC in DNA.

31. (canceled)

32. (canceled)

33. (canceled)
